(19) United States
(12) Patent Application Publication
(10) Pub. No.: US 2026/0207307 A1
(43) Pub. Date: Jul. 23, 2026

(54) SHAPED BLOCK COMPRISING COLLAGEN

(71) Applicant: Datum Dental Ltd., Yavne (IL)

(72) Inventor: Thomas Bayer, Tel Aviv (IL)

(21) Appl. No.: 19/029,753

(22) Filed: Jan. 17, 2025

Related U.S. Application Data
(63) Continuation of application No. 17/041,049, filed on Mar. 27, 2019, now abandoned.

(30) Foreign Application Priority Data
Mar. 27, 2018 (IL) 258392

(51) Int. Cl. A61C 8/00

(52) U.S. Cl. CPC A61C 8/0056

(57) ABSTRACT

This invention is directed to a shaped block, a dental implant abutment comprising an implant, an abutment and a shaped block, wherein the shaped block comprises a dried collagen matrix; and a method of preparation thereof.

### FIELD OF THE INVENTION

This invention is directed to a shaped block, a dental implant abutment comprising an implant, an abutment and a shaped block, wherein the shaped block comprises a dried collagen matrix; and a method of preparation thereof.

### BACKGROUND OF THE INVENTION

Gingival inflammation around dental implants (peri implant mucositis) often leads to an irreversible bone loss (peri implantitis) that if not properly treated, may lead to implant loss. It is estimated to affect about ~30% of all dental implants and is of major concern to patients and doctors.

One of many potential etiological factors for the development of peri implantitis is bacterial colonization within the biologic width i.e. the minimal distance between the implant margin and the alveolar bone crest. It is often correlated to inadequate soft tissue thickness which was shown to increase bone resorption following implant abutment connection. Another widely studied factor is soft tissue seal around implant abutments which is often characterized by fibroblasts and epithelial cells' attachment to titanium or zirconia. This essential attachment may be lost when repeated connection and disconnection of abutments is practiced during implant restorative phase.

The need to augment and reinforce the soft tissue attachment and thickness around dental implants yielded several surgical techniques, mostly based on harvesting dense autogenous connective tissue and implanting it next to implants at risk or in the esthetic zone. Limited surgical skills limit these procedures only to highly skilled surgeons. Hence, there is a need for a solution that involves the use of a medical device that will be effective and easy to use. This device should be able to support soft tissue augmentation and allow formation and maintenance of the biologic width.

This invention provides a shaped block comprising a dried collagen matrix. This block can be shaped in various shapes, e.g. an O-ring and was found to form and augment a connective tissue seal around dental-implant abutments.

### SUMMARY OF THE INVENTION

In one embodiment, this invention is directed to a shaped block comprising a dried cross-linked collagen matrix. In another embodiment, the dried cross-linked collagen matrix further comprising hydroxyapatite, titanium, a pharmaceutically active agent or any combination thereof.

In one embodiment, this invention is directed to a shaped block for use as an add-on device for medical devices, implants, device attachments or any combination thereof, where the shaped block comprises a dried cross-linked collagen matrix. In another embodiment, the dried cross-linked collagen matrix further comprising hydroxyapatite, titanium, a pharmaceutically active agent or any combination thereof.

In one embodiment, this invention is directed to a dental implant abutment comprising an implant, an abutment and a shaped block comprising dried cross-linked collagen matrix; wherein the shaped block is positioned between the abutment and the implant as such that it is in direct contact at least partially with the implant and/or with the abutment. In another embodiment, the positioning of the shaped block within the dental-implant abutment allows a base for soft/hard tissue ingrowth. In another embodiment, sliding motion of shaped block between implant and abutment allows bridging the abutment and implant with the shaped block, giving rise to supported tissue ingrowth.

In one embodiment, this invention is directed to a dental implant abutment for use in the stimulation of bone or soft tissue growth, where the dental implant abutment comprises an implant, an abutment and a shaped block comprising dried cross-linking collagen matrix; wherein the shaped block is positioned between the abutment and the implant as such that it is in direct contact at least partially with the implant; the shaped block of the dental implant abutment is in partial and/or full contact with the bone and/or soft tissue surrounding the dental implant abutment; and the dental implant abutment of the invention provides space and environment for cell ingrowth.

In another embodiment, the shaped block is shaped as an O-ring-like, a sleeve-like or a tube-like (FIG. 2a).

In one embodiment, this invention is directed to a method of preparing a shaped block comprising dried cross-linked collagen matrix, wherein the method comprises:
a. providing a dried cross-linked collagen matrix of this invention; and
b. carving out a shaped block from said dried matrix.

In one embodiment, this invention is directed to a method of preparing a shaped block of the dental implant abutment as described hereinabove, wherein the method comprises:
(i) providing an acidic solution of collagen, followed by neutralization the solution;
(ii) concentrating the solution of step (i); and pouring it into a mold with a pre-designed block shape;
(iii) lyophilizing the concentrated mixture of step (ii), thereby obtaining a dried collagen composition, optionally comprising hydroxyapatite;
(iv) incubating the composition with a crosslinker, a first solvent and optionally with a pharmaceutically active agent;
(v) washing the incubated composition of step (iv) with a second solvent; and
(vi) lyophilizing the washed composition of step (v);
thereby obtaining a shaped block of the dental implant abutment as described hereinabove.

In one embodiment, this invention is directed to a method of preparing a shaped block of the dental implant abutment as described hereinabove, wherein the method comprises:
(i) providing an acidic solution of collagen and a crosslinker, followed by neutralization the solution;
(ii) concentrating the solution of step (i); and pouring it into a mold with a pre designed block shape;
(iii) lyophilizing the concentrated mixture of step (ii), thereby obtaining a dried collagen composition, optionally comprising hydroxyapatite;
(iv) incubating the composition with the crosslinker, a first solvent and optionally with a pharmaceutically active agent;
(v) washing the incubated composition of step (iv) with a second solvent; and
(vi) lyophilizing the washed composition of step (v);
thereby obtaining a shaped block of the dental implant abutment as described hereinabove.

In one embodiment, this invention is directed to a method of preparing a shaped block of the dental implant abutment as described hereinabove, wherein the method comprises:
(i) providing an acidic solution of collagen, followed by neutralization the solution;
(ii) concentrating the solution of step (i); and pouring it into a mold with a pre designed block shape;
(iii) lyophilizing the concentrated mixture of step (ii), thereby obtaining a dried collagen composition, optionally comprising hydroxyapatite;
(iv) incubating the composition with a crosslinker and a first solvent;
(v) optionally adding a pharmaceutically active agent to the incubated composition of step (iv) and washing the resulting solution with a second solvent; and
(vi) lyophilizing the washed composition of step (v);
thereby obtaining a shaped block of the dental implant abutment as described hereinabove.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a dental implant abutment of the invention, comprising an implant, an abutment and a shaped block, denoted by arrows.

FIGS. 2a-2b depict shaped blocks of the invention. FIG. 2a: drawing of such shapes; and FIG. 2b: shaped blocks prepared according to embodiments of the invention.

It will be appreciated that for simplicity and clarity of illustration, elements shown in the Figures have not necessarily been drawn to scale. For example, the dimensions of some of the elements may be exaggerated relative to other elements for clarity. Further, where considered appropriate, reference numerals may be repeated among the Figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Shaped Block and Uses Thereof

In one aspect, the invention provides a shaped block comprising dried collagen matrix. In one embodiment, the dried collagen matrix comprises a crosslinked collagen. In one embodiment, the invention provides a shaped block comprising dried cross-linked collagen matrix. In another embodiment, the shaped block further comprises hydroxyapatite, titanium, pharmaceutically active agent or any combination thereof.

In another aspect, the invention provides a shaped block for use as an add-on element/unit for medical devices, implants, device attachments or any combination thereof, wherein the shaped block comprises a dried cross-linked collagen matrix.

In another embodiment, the shaped block is used as an add-on element/unit for medical devices. In another embodiment, the shaped block is used as an add-on element/unit for implants. In another embodiment, the shaped block is used as an add-on element/unit for device attachments. In another embodiment, any medical device as known in the art can be used. In another embodiment, implants are dental implant bodies. In another embodiment, any dental implant bodies as known in the art can be used. In another embodiment, non-limiting examples for device attachments include prosthetics, shaped screws, shaped plates, shaped wires or any other attachment for bone/jar reconstruction. Each possibility represents a separate embodiment of the invention.

In some embodiments, the term “add-on element/unit” refers to a shaped physical product that can be added to a known, other apparatus, device or product. In other embodiments, the addition is implemented by a person skilled in the art of the invention. In other embodiment, the addition comprises the following non-limiting actions: putting the add-on element/unit on or in proximity to the other device, manipulating them chemically to afford a robust connectivity in between them (e.g. welding, melting and solidification, dissolving and precipitation), manipulating them mechanically (e.g. screwing, rubbing) and any combination thereof. In another embodiment, the add-on unit/element is a shaped block of the invention which is added on a dental implant abutment, on wires, on plates, on screws or on clamps or on any other device, apparatus or product as known in the art. Each possibility represents a separate embodiment of the invention.

In some embodiments, the term “block” refers to some physical extent of a solid matter. In other embodiments, the solid matter is dry and does not comprise any solvent or liquid. In other embodiments, the solid matter comprises at least one sole component. In another embodiment, non-limiting examples of a component include: a chemical compound, small molecule, metal, alloy, composite material, biomaterial, polymer and organometallic complex. In another embodiment, the solid matter comprises more than one component as selected from the foregoing list. In another embodiment, the block comprises a biocompatible material or composition. In another embodiment, the block is a biopolymer or a protein. In other embodiments, the physical attributes of the block afford its design, molding, carving out or engineering as known in the art into a desired shape. In another embodiment, non-limiting examples of attributes of the block include the following characteristics: dense, porous or non-porous, viscous, rigid, soft or moldable. In another embodiment, the block has a porosity of between 10-90%. In another embodiment, the block has a porosity of between 10-20%. In another embodiment, the block has a porosity of between 20-30%. In another embodiment, the block has a porosity of between 30-40%. In another embodiment, the block has a porosity of between 40-50%. In another embodiment, the block has a porosity of between 50-60%. In another embodiment, the block has a porosity of between 60-70%. In another embodiment, the block has a porosity of between 70-80%. In another embodiment, the block has a porosity of between 80-90%. In another embodiment, the block has a porosity of between 10-30%. In another embodiment, the block has a porosity of between 30-50%. In another embodiment, the block has a porosity of between 50-70%. In another embodiment, the block has a porosity of between 70-90%. Each possibility represents a separate embodiment of the invention.

In some embodiments, the term “shaped block” refers to a block which is provided in some shape or form, i.e. designed, engineered, manufactured or prepared as known in the art to provide some two or three-dimensional structure of such block. In other embodiments, non-limiting examples of the two or three-dimensional structure include: square, circle, triangle, o-ring, sleeve, tube, pyramid, box, cuboid, cylinder, conc, prism and any other structure containing a hole within. In other embodiments, any possible design, engineering, manufacturing or preparation process can be applied in order to provide the shaped block.

In some embodiments, the term “collagen” refers to a biopolymer organized in a fibrillar networks or other non-fibrillar superstructures, and it is the main component of connective tissue within the human or numerous animals' body. Numerous types of collagen are known and found naturally. Non-limiting examples include types I-V. In some embodiments, collagen has a fibrillar (such as Type I) or non-fibrillar structure. Each possibility represents a separate embodiment of the invention.

In one embodiment, the collagen used in the methods, uses and shape blocks of this invention refer to native collagen, fibrillar collagen, fibrillar atelopeptide collagen, lyophilized collagen, collagen obtained from animal sources, human collagen, recombinant collagen, pepsinized collagen, reconstituted collagen and any combination thereof. In another embodiment, the collagen includes fibrillar collagen reconstituted from monomolecular atelopeptide collagen. In another embodiment, the collagen is atelopeptide fibrillar collagen obtained by reconstituting monomolecular atelopeptide collagen obtained by proteolytic digestion of native collagen. Each possibility represents a separate embodiment of the invention.

In some embodiments, the term “cross-linked collagen” refers to a covalent network comprising biopolymer chains of collagen connected covalently and inter-molecularly with crosslinkers.

In some embodiments, the term “crosslinkers” refers to small molecules or polymers comprising at least two ends that can covalently connect polymeric/oligomeric chains and thereby crosslink these chains.

In some embodiments the collagen is cross-linked by a sugar. In another embodiment, the sugar is selected from the group consisting of glycerose (glyceraldehyde), threose, erythrose, lyxose, xylose, arabinose, ribose, allose, altrose, glucose, mannose, gulose, idose, galactose, talose and any combination thereof. Each possibility represents a separate embodiment of the invention.

In another embodiment, the sugar is a disaccharide. Each possibility represents a separate embodiment of the invention.

In another embodiment, the disaccharide is selected from the group consisting of maltose, lactose, sucrose, cellobiose, gentiobiose, melibiose, turanose, trehalose and any combination thereof. Each possibility represents a separate embodiment of the invention.

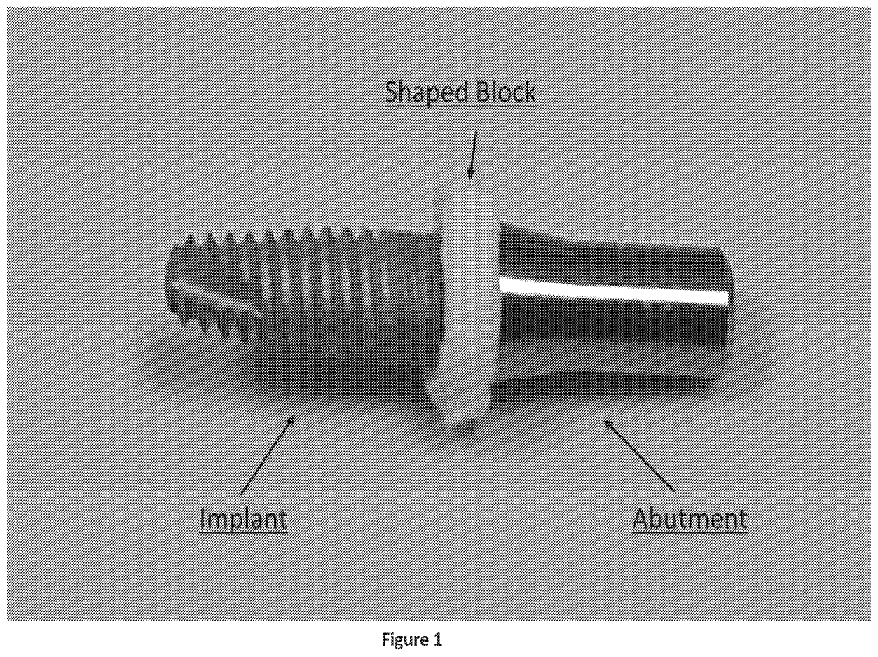

In another aspect, the invention provides a dental implant abutment (FIG. 1) comprising an implant, an abutment and a shaped block as described herein. In one embodiment, this invention provides a dental implant abutment comprising an implant, an abutment and a shaped block comprising dried cross-linked collagen matrix, wherein the shaped block is positioned between the abutment and the implant as such that it is in contact at least partially with the implant and/or with the abutment. In another embodiment, the positioning of the shaped block within the dental-implant abutment allows a base for soft/hard tissue ingrowth. In another embodiment, sliding motion of shaped block between implant and abutment allows bridging the abutment and implant with the shaped block, giving rise to supported tissue ingrowth. In another embodiment, the shaped block of the dental implant abutment is in partial and/or full contact with the bone and/or soft tissue surrounding the dental implant abutment.

In another embodiment, the implant is partially in contact with the shaped block. In another embodiment, the implant is covered completely by the shaped block. In one embodiment, any implant of the dental implant abutment of the invention can be utilized, provided or made, in any method as known in the art. In another embodiment, the implant is made from a material comprising polymers, ceramics, metals (e.g. Ti, Zr) or alloys or any combination thereof. In another embodiment, the shape/size of the implant comprises conical, cylindrical, plate, wire thread, hole(s)-containing shapes or any combination thereof. In one embodiment, any abutment of the dental implant abutment of the invention can be utilized, provided or made in any method as known in the art. In another embodiment, the abutment is made from a material comprising polymers, ceramics, metals (e.g. Ti, Zr) or alloys or any combination thereof. In another embodiment, the shape/size of the abutment comprises cylinders, cones, cubes or any combination thereof. In some embodiments, non-limiting examples of polymers for abutments and/or implants include: polyurethane, polymethylmethacrylate, polysiloxanes, polylactic acid, polyacrylamides, any combination thereof and any other biocompatible polymer. In some embodiments, non-limiting examples of ceramics for abutments and/or implants include: zirconia, alumina, titania, calcium phosphates, any combination thereof and any other biocompatible ceramic. In some embodiments, non-limiting examples of metals or alloys for abutments and/or implants include: Ti, Zr, Ni, NiTi, any combination thereof and any other biocompatible metal or alloy. Each possibility represents a separate embodiment of the invention.

In yet another aspect, the dental implant abutment of the invention is used in the stimulation of bone or soft tissue growth. In one embodiment, the dental implant abutment of the invention provides space and environment for cell ingrowth. The soft but fitting nature of the material allows a closed fitting onto the surface of the abutment and the implant as well as onto the surrounding soft and bone tissue. This proximity in combination with the bone and soft tissue conductive properties allows a fast and effective incorporation of the abutment.

In another embodiment, the shaped block further comprises hydroxyapatite, titanium, pharmaceutically active agent or any combination thereof.

In other embodiments, non-limiting examples for the pharmaceutically active agent include antibacterial agents, antifungal agents, anti-septic agents, anti-inflammatory agents, antibiotic agents, vitamins or any combination thereof. In another embodiment, any agent as known in the art within the foregoing list can be utilized. Each possibility represents a separate embodiment of the invention.

In other embodiments, non-limiting examples for the active agent include antibacterial agents, antifungal agents, anti-septic agents, anti-inflammatory agents, antibiotic agents, vitamins and vitamers and any combination thereof. In another embodiment, any agent as known in the art within the foregoing list can be utilized. Each possibility represents a separate embodiment of the invention.

In another embodiment, non-limiting examples of antibacterial agents include: Amikacin, Gentamicin, Kanamycin, Neomycin, Netilmicin, Tobramycin, Paromomycin, Arbekacin, Plazomicin, Streptomycin, Apramycin, Geldanamycin, Herbimycin, Loracarbef, Faropenem, Ertapenem, Doripenem, Imipenem, Meropenem, Cefazolin, Cefacetrile, Cefadroxil, Cephalexin, Cefaloglycin, Cefalonium, Cefaloridine, Cefalotin, Cefapirin, Cefatrizine, Cefazedone, Cefazaflur, Cefradine, Cefroxadine, Ceftezole, Cefaclor, Cefamandole, Cefminox, Cefonicid, Ceforanide, Cefotiam, Cefprozil, Cefbuperazone, Cefuroxime, Cefuzonam, Cephamycin, Ccfoxitin, Cefotctan, Cefmctazole, Carbacephem, Cefixime, Ceftazidimc, Ceftriaxone, Cefcapene, Cefdaloxime, Cefdinir, Cefditoren, Cefetamet, Cefmenoxime, Cefodizime, Cefoperazone, Cefotaxime, Cefpimizole, Cefpiramide, Cefpodoxime, Cefsulodin, Cefteram, Ceftibuten, Ceftiolene, Ceftizoxime, Oxacephem, Cefepime, Cefozopran, Cefpirome, Cefquinome, Ceftiofur, Cefquinome, Cefovecin, CXA-101, Ceftaroline, Ceftobiprole, Clindamycin, Lincomycin, Azithromycin, Clarithromycin, Dirithromycin, Erythromycin, Roxithromycin, Troleandomycin, Telithromycin, Spectinomycin, Solithromycin, Aztreonam, Furazolidone, Nitrofurantoin, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin, iprofloxacin, Enoxacin, Gatifloxacin, Levofloxacin, Lomefloxacin, Moxifloxacin, Nalidixic acid, Levonadifloxacin, Norfloxacin, Ofloxacin, Trovafloxacin, Grepafloxacin, Sparfloxacin, Temafloxacin, Delafloxacin, Mafenide, Sulfonamidochrysoidine, Sulfacetamide, Sulfadiazine, Sulfamethizole, Sulfamethoxazole, Sulfasalazine, Sulfisoxazole, Trimethoprim, emeclocycline, Doxycycline, Minocycline, Oxytetracycline, Tetracycline, Tigecycline, Tedizolid, Linezolid, Ranbezolid, Torezolid, Radczoli, any combination thereof and any pharmaceutically acceptable salt thereof. Each possibility represents a separate embodiment of the invention.

In another embodiment, non-limiting examples of antifungal agents include: terbinafine, naftifine, amphotericin B, butenafine, chloroxylenol, ciclopirox, flucytosine, caspofungin, griseofulvin, clotrimazole, fluconazole, itraconazole, ketoconazole, miconazole, oxiconazole, nystatin, undecylenic acid, any combination thereof and any pharmaceutically acceptable salt thereof. Each possibility represents a separate embodiment of the invention.

In another embodiment, non-limiting examples of anti-septic agents include: naftifine, tolnaftate, mediocidin, candicidin, trichomycin, hamycin, aurefungin, ascosin, ayfattin, azacolutin, trichomycin, levorin, heptamycin, candimycin, griseofulvin, pradimicins, benanomicin; ambisome; nikkomycin Z; flucytosine, perimycin, any combination thereof and any pharmaceutically acceptable salt thereof. Each possibility represents a separate embodiment of the invention.

In another embodiment, non-limiting examples of anti-inflammatory agents include: aspirin, ibuprofen, naproxen, celecoxib, diclofenac, ketoprofen, ketorolac, oxaprozin, salsalate, sulindac, any combination thereof and any pharmaceutically acceptable salt thereof. Each possibility represents a separate embodiment of the invention.

In another embodiment, non-limiting examples of antibiotics include penicillin, cephalosporin, ciprofloxacin, erythromycin, any combination thereof and any pharmaceutically acceptable salt thereof. Each possibility represents a separate embodiment of the invention.

In another embodiment, non-limiting examples of vitamins include: vitamin a, retinol, retinal, carotenoid, vitamin B1, thiamine, vitamin B2, riboflavin, vitamin B3, niacin, niacinamide, nicotinamide, riboside, vitamin B5, pantothenic acid, vitamin B6, pyridoxine, pyridoxamine, pyridoxal, vitamin B7, biotin, vitamin B9, folates, vitamin B12, cyanocobalamin, hydroxocobalamin, methylcobalamin, adenosylcobalamin, vitamin C, ascorbic acid, vitamin D, cholecalciferol (D3), ergocalciferol (D2), vitamin E, tocopherols, tocotrienols, vitamin K, phylloquinone, menaquinones, any combination thereof and any pharmaceutically acceptable salt thereof. Each possibility represents a separate embodiment of the invention.

In some embodiments, the term “hydroxyapatite” refers to the naturally occurring calcium mineral with the formula of Ca₅(PO₄)₃(OH) or Ca₁₀(PO₄)₆(OH)₂. In other embodiments, hydroxyapatite can be prepared or obtained via any known method in the art. In another embodiment, bulk or nanoparticulate hydroxyapatite is utilized and/or prepared. Each possibility represents a separate embodiment of the invention.

In some embodiments, the term “titanium” refers to a titanium metal or alloy. In other embodiments, titanium is alloyed, with the non-limiting examples of Zr and/or Ni. In another embodiment the titanium is biocompatible. In another embodiment, the titanium is a biocompatible titanium alloy. In other embodiments, the titanium metal is of any shape or form as known in the art. In another embodiment, the titanium or alloyed titanium may have a shape memory function. In another embodiment, the titanium metal shape or form comprises bulk metal, surface thereof or nanoparticulate matter. In another embodiment, bulk metal is rod shaped, plate shaped, cube shaped or shaped in any other physical form as known in the art. Each possibility represents a separate embodiment of the invention.

In some embodiments, the term “nanoparticulate matter” (e.g. hydroxyapatite or titanium) refers to a matter which has at least one physical nanometric dimension. In another embodiment, nanoparticulate matter is shaped as nanoparticles, nanospheres, nanocubes, nanoplates, nanoribbons, nanowires, nanorods or in any other nanometric shape as known in the art.

Method of Preparing a Shaped Block of this Invention

In one further aspect, this invention is directed to a method of preparing a shaped block comprising dried cross-linked collagen matrix of this invention wherein the method comprises:
a. providing a dried cross-linked collagen matrix of this invention; and
b. carving out a shaped block from said dried matrix.

In another embodiment, the dried cross-linked collagen matrix comprises hydroxyapatite. In another embodiment, the dried cross-linked collagen matrix comprises titanium. In another embodiment, the cross-linked collagen matrix comprises a pharmaceutically active agent. In another embodiment, the dried cross-linked collagen matrix comprises hydroxyapatite, titanium, a pharmaceutically active agent or any combination thereof.

In one embodiment, this invention is directed to a method of preparing a dried cross-linked collagen matrix wherein the matrix comprises a cross-linked collagen and the method comprises:
(i) providing an acidic solution of collagen, followed by neutralization of the solution;
(ii) concentrating the solution of step (i);
(iii) lyophilizing the concentrated mixture of step (ii), thereby obtaining a dried collagen composition;
(iv) incubating the composition with a crosslinker and a first solvent;
(v) washing the incubated composition of step (iv) with a second solvent;
(vi) lyophilizing the washed composition of step (v);
thereby obtaining a dried cross-linked collagen matrix of this invention.

In one embodiment, this invention is directed to a method of preparing a dried cross linked collagen matrix, wherein the matrix comprises a cross linked collagen and optionally hydroxyapatite, titanium, a pharmaceutically active agent or any combination thereof and the method comprises:
(i) providing an acidic solution of collagen, followed by neutralization of the solution wherein the neutralization solution optionally comprises hydroxyapatite;
(ii) concentrating the solution of step (i) and optionally adding titanium;
(iii) lyophilizing the concentrated mixture of step (ii), thereby obtaining a dried collagen composition;
(iv) incubating the composition with a crosslinker, a first solvent and optionally adding a pharmaceutically active agent;
(v) washing the incubated composition of step (iv) with a second solvent;
(vi) lyophilizing the washed composition of step (v);
   thereby obtaining a dried cross linked collagen matrix of this invention.

In one embodiment, this invention is directed to a method of preparing a dried cross linked collagen matrix, wherein the matrix comprises a cross linked collagen and hydroxyapatite and the method comprises:
(i) providing an acidic solution of collagen, followed by neutralization of the solution, wherein the neutralization solution comprises hydroxyapatite;
(ii) concentrating the solution of step (i);
(iii) lyophilizing the concentrated mixture of step (ii), thereby obtaining a dried collagen composition;
(iv) incubating the composition with a crosslinker and a first solvent;
(v) washing the incubated composition of step (iv) with a second solvent;
(vi) lyophilizing the washed composition of step (v);
   thereby obtaining a dried cross-linked collagen matrix of this invention.

In one embodiment, this invention is directed to a method of preparing a dried cross linked collagen matrix, wherein the matrix comprises a cross linked collagen and a pharmaceutically active agent and the method comprises:
(i) providing an acidic solution of collagen, followed by neutralization of the solution;
(ii) concentrating the solution of step (i);
(iii) lyophilizing the concentrated mixture of step (ii), thereby obtaining a dried collagen composition;
(iv) incubating the composition with a crosslinker, a first solvent and a pharmaceutically active agent;
(v) washing the incubated composition of step (iv) with a second solvent;
(vi) lyophilizing the washed composition of step (v);
   thereby obtaining a dried cross-linked collagen matrix of this invention.

In one embodiment, this invention is directed to a method of preparing a dried cross linked collagen matrix, wherein the matrix comprises a cross linked collagen, titanium and a pharmaceutically active agent and the method comprises:
(i) providing an acidic solution of collagen, followed by neutralization of the solution;
(ii) concentrating the solution of step (i), adding titanium and
(iii) lyophilizing the concentrated mixture of step (ii), thereby obtaining a dried collagen composition;
(iv) incubating the composition with a crosslinker, a first solvent and a pharmaceutically active agent;
(v) washing the incubated composition of step (iv) with a second solvent;
(vi) lyophilizing the washed composition of step (v);
   thereby obtaining a dried cross-linked collagen matrix of this invention.

In another embodiment, compressing steps (applying mechanical pressure using a specialized equipment) are applied in addition to, or instead of the lyophilization steps.

In some embodiments, the carving out is done by any method as known in the art. In another embodiment, the carving out of the method of the invention is done by a CNC (Computer numerical control) machine, a laser cutting machine, a waterjet cutter, a driller or an abrasive device. Each possibility represents a separate embodiment of the invention.

In some embodiments, the shaped block of the invention is provided in various shapes. In some embodiments, the blocks are shaped to allow an easy fitting of the block onto a dental abutment and to allow at least partial coverage of a dental implant once the abutment is placed into the implant. In another embodiment, the block is shaped as O-ring-like, a sleeve-like or a tube-like. Each possibility represents a separate embodiment of the invention.

Method of Preparing a Shaped Block of this Invention Using a Mold

In one additional aspect, this invention is directed to a method of preparing a shaped block comprising dried cross-linked collagen matrix, wherein the matrix comprises a cross linked collagen, optionally hydroxyapatite and optionally a pharmaceutically active agent and the method comprises:
(i) providing an acidic solution of collagen, followed by neutralization of the solution;
(ii) concentrating the solution of step (i); and pouring it into a mold with a pre designed block shape;
(iii) lyophilizing the concentrated mixture of step (ii), thereby obtaining a dried collagen composition, optionally comprising hydroxyapatite;
(iv) incubating the composition with a crosslinker, a first solvent and optionally with a pharmaceutically active agent;
(v) washing the incubated composition of step (iv) with a second solvent;
(vi) lyophilizing the washed composition of step (v);
   thereby obtaining a shaped block of this invention. In another embodiment, the neutralization solution of step (i) optionally comprises hydroxyapatite.

In one embodiment, this invention is directed to a method of preparing a shaped block comprising dried cross-linked collagen matrix, wherein the matrix comprises a cross linked collagen, optionally hydroxyapatite and optionally a pharmaceutically active agent and the method comprises:
(i) providing an acidic solution of collagen and a crosslinker, followed by neutralization of the solution;
(ii) concentrating the solution of step (i); and pouring it into a mold with a pre designed block shape;
(iii) lyophilizing the concentrated mixture of step (ii), thereby obtaining a dried collagen composition, optionally comprising hydroxyapatite;
(iv) incubating the composition with the crosslinker, a first solvent and optionally with a pharmaceutically active agent;
(v) washing the incubated composition of step (iv) with a second solvent; and
(vi) lyophilizing the washed composition of step (v);
   thereby obtaining a shaped block of this invention. In another embodiment, the neutralization solution of step (i) optionally comprises hydroxyapatite.

In one embodiment, this invention is directed to a method of preparing a shaped block comprising dried cross-linked collagen matrix, wherein the matrix comprises a cross-linked collagen, optionally hydroxyapatite, optionally a pharmaceutically active agent and the method comprises:
(i) providing an acidic solution of collagen, followed by neutralization of the solution;
(ii) concentrating the solution of step (i); and pouring it into a mold with a pre designed block shape;
(iii) lyophilizing the concentrated mixture of step (ii), thereby obtaining a dried collagen composition, optionally comprising hydroxyapatite;
(iv) incubating the composition with a crosslinker and a first solvent;
(v) optionally adding a pharmaceutically active agent to the incubated composition of step
(iv) and washing the resulting solution with a second solvent; and
(vi) lyophilizing the washed composition of step (v);
   thereby obtaining a shaped block of this invention. In another embodiment, the neutralization solution of step (i) optionally comprises hydroxyapatite.

In one embodiment, this invention is directed to a method of preparing a shaped block comprising dried cross-linked collagen matrix, wherein the matrix comprises a cross-linked collagen, optionally hydroxyapatite, optionally titanium and optionally a pharmaceutically active agent and the method comprises:
(i) providing an acidic solution of collagen, followed by neutralization of the solution
(ii) concentrating the solution of step (i), optionally adding titanium; and pouring it into a mold with a pre designed block shape;
(iii) lyophilizing the concentrated mixture of step (ii), thereby obtaining a dried collagen composition, optionally comprising hydroxyapatite;
(iv) incubating the composition with a crosslinker, a first solvent and optionally with a pharmaceutically active agent;
(v) washing the incubated composition of step (iv) with a second solvent;
(vi) lyophilizing the washed composition of step (v);
   thereby obtaining a shaped block of this invention.

In another embodiment, compressing steps (applying mechanical pressure using a specialized equipment) are applied in addition to, or instead of the lyophilization steps.

In another embodiment, the mold is cooled to a freezing temperature of between −10° C. to −190° C. prior to the lyophilization step (iii). In another embodiment, the mold is cooled to a freezing temperature of between −10 to −80° C. for a period of between 0.5 to 24 hours followed by a lyophilization step (iii). In another embodiment, the cooling is carried out for between 0.5-24 hours. In another embodiment, the cooling is carried out for between 0.5-1 hours. In another embodiment, the cooling is carried out for between 1-2 hours. In another embodiment, the cooling is carried out for between 2-5 hours. In another embodiment, the cooling is carried out for between 5-10 hours. In another embodiment, the cooling is carried out for between 10-24 hours. Each possibility represents a separate embodiment of the invention.

In another embodiment, the mold is shaped to afford the desired block shape, using a method comprising 3D printing, cast molding or any combination thereof. Each possibility represents a separate embodiment of the invention.

Method of Preparing a Shaped Block of this Invention Via Granulates

In one embodiment, this invention is directed to a method of preparing a shaped block comprising dried cross-linked collagen matrix, wherein the matrix comprises a cross linked collagen, optionally hydroxyapatite, optionally titanium and optionally a pharmaceutically active agent and the method comprises:
(i) providing an acidic solution of collagen, followed by neutralization of the solution
(ii) concentrating the solution of step (i) and optionally adding titanium;
(iii) incubating the composition with a crosslinker, a first solvent and optionally adding a pharmaceutically active agent;
(iv) washing the incubated composition of step (iv) with a second solvent;
(v) homogenizing, casting and milling the composition to obtain granulates of cross linked collagen;
(vi) wetting the granulates of step (v) by the first or second solvent and carving out to obtain a shaped block of this invention.

In some embodiments, the shaped block and/or dried cross linked collagen matrix prepared via the methods of this invention as described hereinabove—are used in the dental implant abutment of this invention, also described above; and/or they (block/matrix) are part of such dental implant abutment.

In some embodiments, the shaped block or blocks obtained by the methods of this invention are milled to form granulates and then wetted by a first or second solvent and carved out to obtain a shaped block comprising cross-linked collagen matrix. In another embodiments, the size of the granulates are between 1 to 2000 microns.

In some embodiments, the collagen used in the methods of this invention within the solution of step “(i)” is selected from the following non-limiting examples including: native collagen, fibrillar collagen, fibrillar atelopeptide collagen, lyophilized collagen, collagen obtained from animal sources, human collagen, recombinant collagen, pepsinized collagen, reconstituted collagen and any combination thereof. In another embodiment, the collagen includes fibrillar collagen reconstituted from monomolecular atelopeptide collagen. In another embodiment, the collagen is atelopeptide fibrillar collagen obtained by reconstituting monomolecular atelopeptide collagen obtained by proteolytic digestion of native collagen. Each possibility represents a separate embodiment of the invention.

In another embodiment, the neutralization solution comprises a base or a buffer. In another embodiment, the neutralization solution further comprises hydroxyapatite. In another embodiment the buffer is selected from a phosphate buffered saline, NaHCO₃/Na₂CO₃ buffer, tris buffer or a tricine buffer or any other buffer that maintains a neutral pH. In another embodiment, the acidic solution comprises HCl, acetic acid, nitric acid, citric acid, sulfuric acid, phosphoric acid or any other acid as known in the art. In another embodiment, the basic solution comprises NaOH, KOH, NaHCO₃, Na₂CO₃, Na₂HPO₄ or any other base as known in the art.

In some embodiments, the term “neutral pH” refers to a range of pHs which resembles the physiological pH in biological body and/or system; and it's defined between 6.5-7.5. In some other embodiments, a neutral pH is between 6.5-6.7. In some other embodiments, a neutral pH is between 6.7-6.9. In some other embodiments, a neutral pH is between 6.9-7.1. In some other embodiments, a neutral pH is between 7.1-7.3. In some other embodiments, a neutral pH is between 7.3-7.5. In some other embodiments, a neutral pH is between 7.1-7.2. In some other embodiments, a neutral pH is between 7.2-7.3. In some other embodiments, a neutral pH is between 7.3-7.4. In some other embodiments, a neutral pH is between 7.4-7.5.

In one embodiment, the concentrating step is done by centrifugation. In another embodiment, the centrifugation is carried out at a rate of between 50-20,000 RPM (rounds per minute). In another embodiment, centrifugation is carried out at a rate of between 50-100 RPM. In another embodiment, centrifugation is carried out at a rate of between 100-1,000 RPM. In another embodiment, centrifugation is carried out at a rate of between 1,000-5,000 RPM. In another embodiment, centrifugation is carried out at a rate of between 5,000-10,000 RPM. In another embodiment, centrifugation is carried out at a rate of between 10,000-20,000 RPM. In another embodiment, centrifugation is carried out for between 1-120 minutes. In another embodiment, centrifugation is carried out for between 1-5 minutes. In another embodiment, centrifugation is carried out for between 5-10 minutes. In another embodiment, centrifugation is carried out for between 10-20 minutes. In another embodiment, centrifugation is carried out for between 20-50 minutes. In another embodiment, centrifugation is carried out for between 50-100 minutes. In another embodiment, centrifugation is carried out for between 100-120 minutes. Each possibility represents a separate embodiment of the invention.

In another embodiment, the lyophilization of step (iii) is carried out for between 1-48 hours. In another embodiment, lyophilization of step (iii) is carried out for between 1-2 hours. In another embodiment, lyophilization of step (iii) is carried out for between 2-5 hours. In another embodiment, lyophilization of step (iii) is carried out for between 5-10 hours. In another embodiment, lyophilization of step (iii) is carried out for between 10-24 hours. In another embodiment, lyophilization of step (iii) is carried out for between 24-48 hours. Each possibility represents a separate embodiment of the invention.

Following the lyophilization, a dried collagen composition of this invention is obtained. The dried composition is incubated with a crosslinker, a first solvent and optionally with a pharmaceutically active agent. The incubated composition is further washed with a second solvent and lyophilized to obtain the block collagen composition.

In another embodiment, the first and second solvents are the same or different and selected form any solvent as known in the art. In another embodiment, the solvent is selected from a group comprising: water, ethanol, saline, methanol, phosphate buffer saline or any combination thereof.

In another embodiment, the crosslinker can be any crosslinking agent as known in the art. In another embodiment, the crosslinker is a sugar. In another embodiment, the sugar is a compound represented by at least one of the following formulae I or II:

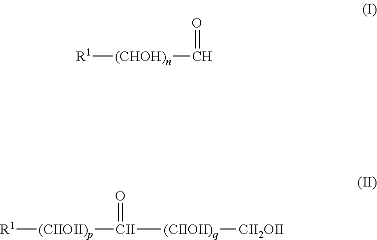

wherein:
R¹ is H or alkyl or alkenyl, an amino acid moiety, a peptide moiety, a saccharide moiety, a purine or a pyrimidine moiety, a phosphorylated purine or pyrimidine moiety;
n is an integer between 2-9, and
p and q are each independently an integer between 0-8, and the sum of p and q is at least 2 and not more than 8.

In another embodiment, the term “alkyl” group refers to a saturated aliphatic hydrocarbon, including straight-chain or branched-chain. In one embodiment, alkyl group is linear or branched. In another embodiment, alkyl is optionally substituted linear or branched. In one embodiment, the alkyl group has between 1-20 carbons. In one embodiment, the alkyl group has between 1-10 carbons. In one embodiment, the alkyl group has between 2-10 carbons. In one embodiment, the alkyl group has between 1-6 carbons. In one embodiment, the alkyl group has between 2-8 carbons. In another embodiment, non-limiting examples of alkyl groups include methyl, ethyl, propyl, isopropyl, isobutyl, butyl, pentyl, 3-pentyl, hexyl heptyl, octyl and hexadecyl. In another embodiment, the alkyl group is optionally substituted by one or more halogens, hydroxides, alkoxides, carboxylic acids, phosphates, phosphonates, sulfates, sulfonates amidates, cyanates, and a nitro group. Each possibility represents a separate embodiment of the invention.

In another embodiment, the term “alkenyl” group refers to an alkyl groups as described herein, having at least one carbon carbon double bond, including straight-chain and branched-chain groups. In one embodiment, the alkene has one double bond. In another embodiment, the alkene has more than one double bond. In another embodiment, the alkene has between 2-6 double bonds, each possibility represents a separate embodiment of this invention. In one embodiment, the alkene has 2-20 carbons. Non-limiting examples include ethylenyl, propylenyl, 2-methylpropyl-1-enyl and butenyl, each possibility represents a separate embodiment of this invention.

In another embodiment, the term “amino acid” refers to an organic compound containing amine (—NH₂) and carboxyl (—COOH) functional groups, along with a side chain specific to each amino acid. In another embodiment, any amino acid as known in the art can be utilized. In another embodiment, amino acid is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine.

In another embodiment, the term “peptide” refers to short chains of amino acids linked covalently via amide (peptide; —C(O)—N(H)—) bonds. In another embodiment, a peptide comprises between 2-20 amino acids. In another embodiment, a peptide is a dipepetide. In another embodiment, a peptide is a tripepetide. In another embodiment, a peptide is a tetrapepetide. In another embodiment, a peptide is a pentapepetide. In another embodiment, a peptide is a hexapepetide.

In another embodiment, the term “saccharide” refers to the group comprising sugars as described herein, cellulose and starch.

In another embodiment, the term “purine” refers to a heterocyclic aromatic organic compound that consists of a pyrimidine ring fused to an imidazole ring. Non limiting examples of purines include: purine, adenin, guanine, hypoxanthine, xanthine, theobromine, caffeine, uric acid and isoguanine.

In another embodiment, the term “pyrimidine” refers to a heterocyclic aromatic organic compound similar to pyridine but has an additional nitrogen within the aromatic ring so nitrogens are found in position 1, 3 of the ring. Non-limiting examples of purines include: cytosine, thymine and uracil.

In another embodiment, the term “a phosphorylated purine or pyrimidine” refers to a purine or pyrimidine as described herein, wherein the purine or pyrimidine is connected to a phosphoryl group (the chemical entity PO₃^{x-}; “x” denoting any possible protonation state).

In another embodiment, the sugar is a naturally occurring reducing sugar.

In another embodiment, the sugar is a diose, a triose, a tetrose, a pentose, a hexose, a septose, an octose, a nanose, or a decose. Each possibility represents a separate embodiment of the invention.

In another embodiment, the sugar is selected from the group consisting of glycerose (glyceraldehyde), threose, erythrose, lyxose, xylose, arabinose, ribose, allose, altrose, glucose, mannose, gulose, idose, galactose, talose and any combination thereof. Each possibility represents a separate embodiment of the invention.

In another embodiment, the sugar is a disaccharide. Each possibility represents a separate embodiment of the invention.

In another embodiment, the disaccharide is selected from the group consisting of maltose, lactose, sucrose, cellobiose, gentiobiose, melibiose, turanose, trehalose and any combination thereof. Each possibility represents a separate embodiment of the invention.

Following the crosslinking and the washing steps with a first and second solution the composition is lyophilized. In another embodiment, the lyophilization of step (vi) is carried out for between 24 to 72 hrs and the block composition is obtained. In one embodiment, the block collagen composition is prepared using a mold with a pre-designed shape, thereby, a block shape collagen composition is obtained. In another embodiment, a mold is not used and a block collagen composition is obtained which is further carved out to a desired block shape collagen composition.

In some other embodiments, the designed mold and the carving out is planned, designed and/or engineered via computer-aided design (CAD) and/or computer-aided manufacturing (CAM) methods and software as known in the art of the invention. Each possibility represents a separate embodiment of the invention.

In another embodiment, the carving out of the method of the invention is done by any method as known in the art. In another embodiment, the carving out of the method of the invention is done by a CNC (Computer numerical control) machine, a laser cutting machine, a waterjet cutter, a driller or an abrasive device. Each possibility represents a separate embodiment of the invention.

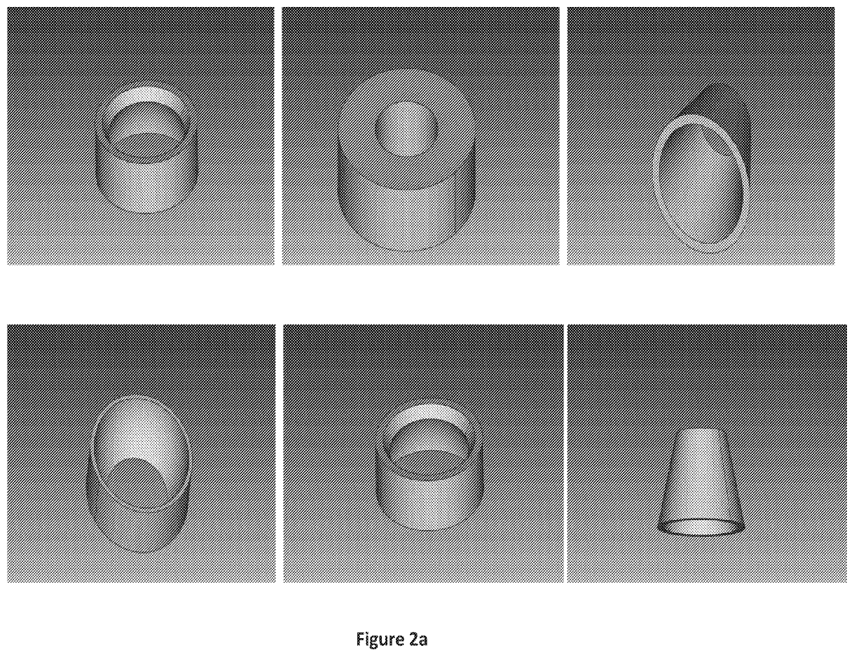

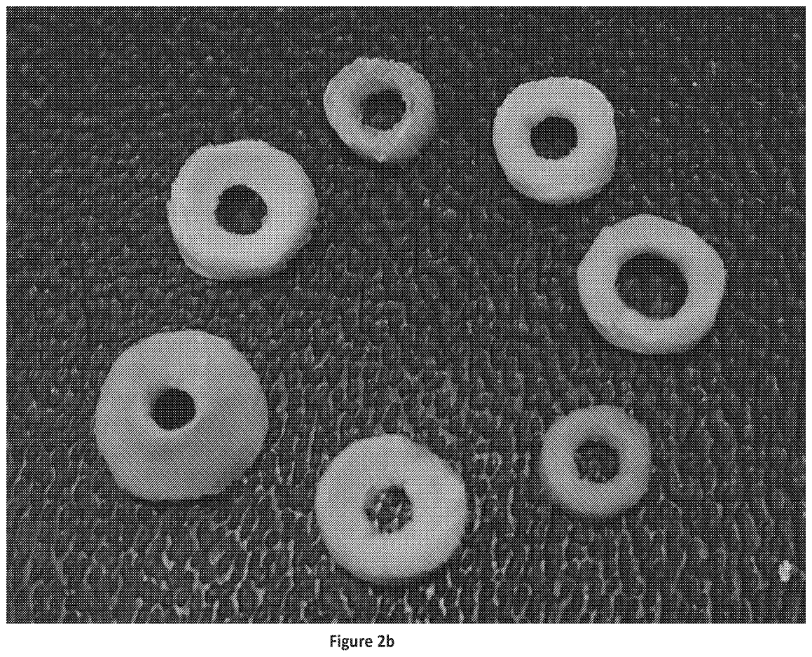

In some embodiments, the shaped block of the invention is provided in various shapes. In some embodiments, the blocks are shaped to allow an easy fitting of the block onto a dental abutment and to allow at least partial coverage of a dental implant once the abutment is placed into the implant. In another embodiment, the block is shaped as O-ring-like, a sleeve-like or a tube-like (FIG. 2a). Each possibility represents a separate embodiment of the invention.

The following examples are presented in order to more fully illustrate the preferred embodiments of the invention. They should in no way, however, be construed as limiting the broad scope of the invention.

### EXAMPLES

### Example 1

### Crosslinking of Collagen

A solution of molecular purified pepsinized Porcine Type I collagen (1-10 milligram/milliliter), prepared from Porcine tendons (commercially available from Pel-Freez, AR, U.S.A), was dissolved in 0.01M HCl and maintained at 4° C. It was neutralized by 0.1M NaOH to pH 7.2-7.4, poured into an appropriate mold, and incubated for 24 hours at a temperature ranging between 20-38° C. The matrix which is produced is then compressed by a piston to remove excess solution. The resulting collagen membranes were incubated for 11 days in PBS.

The fibrillated collagen was concentrated by centrifugation at 3000 rpm. All centrifugations (unless specifically stated otherwise) were done using a model RC5C centrifuge with a SORVALL SS-34 rotor commercially available from SORVALL® Instruments DUPONT, USA. The fibrillated collagen concentration after centrifugation was brought to approximately 35 mg/mL by the use of 10 millimolar phosphate buffer solution (PBS pH 7.36). The mixture was poured into a stainless steel tray. The tray was transferred into the lyophilizer (Freeze dryer model FD 8 commercially available from Heto Lab Equipment DK-3450 Allerød, Denmark), pre-frozen for eight hours and lyophilized for 24 hours. The condenser temperature was −80° C. The shelf temperature during pre-freezing was −40° C. The shelf temperature during lyophilization was +35° C. and the vacuum during lyophilization was approximately 0.01 bar.

200 mL of a solution containing 120 mL absolute ethanol (commercially available from Merck, Germany), 80 mL PBS buffer solution (10 mM, pH 7.36) and 3 gram of D(−) ribose (commercially available as Catalogue No. R7500 from Sigma, USA) were added to the dried (lyophilized) fibrillated collagen and incubated at 37° C. for 11 days to perform the ribose cross-linking of the collagen structure. The ribose cross-linked collagen products were washed exhaustively with DI water and lyophilized, using the same conditions as described above.

### Example 2

### Crosslinking of Collagen-Hydroxyapatite

A solution containing purified collagen consists of atelocollagen monomers [pepsinized type I collagen (~3 mg/mL)], dissolved in 0.05 M acetic acid and maintained at 4° C. was mixed with a slurry of 0.1 M NaOH containing hydroxyapatite (collagen/HA ratio: 95:5-70:30) and brought to a neutral pH. Then the solution was incubated under constant stirring for 24 hours at a temperature ranging between 20-37° C. The fibrillated collagen/HA mixture was concentrated by centrifugation at 3000 rpm for 15 min. The fibrillated collagen concentration after centrifugation was brought to approximately 35 mg/mL by the use of 10 millimolar phosphate buffer solution (PBS pH 7.36). The mixture was homogenized for 10 min at 100m rpm with Planetary Centrifugal Mixer (“THINKY MIXER” ARE-500; THINKY CORPORATION, Japan) poured into a stainless steel tray. The tray was transferred into the lyophilizer (Freeze dryer model FD 8 commercially available from Heto Lab Equipment DK-3450 Allerød, Denmark), pre-frozen for eight hours and lyophilized for 24 hours. The condenser temperature was −80° C. The shelf temperature during pre-freezing was −40° C. The shelf temperature during lyophilization was +35° C. and the vacuum during lyophilization was approximately 0.01 bar.

200 mL of a solution containing 120 mL absolute ethanol (commercially available from Merck, Germany), 80 mL PBS buffer solution (10 mM, pH 7.36) and 3 gram of DL-glyceraldehyde (commercially available from Biosynth, Switzerland) were added to the dried (lyophilized) fibrillated collagen and incubated at 37° C. for 11 days to perform the ribose cross-linking of the collagen structure. The ribose cross-linked collagen products were washed exhaustively with DI water and lyophilized, using the same conditions as described above.

### Example 3

### Carving Out Collagen Blocks

Blocks of the dried collagen matrix have been used to carve out desired shapes. These shapes were of O-ring-like appearance or sleeve-like or tube-like (FIG. 2b). These structures easily fit onto an abutment and partially or fully covering the implant once the abutment is placed onto the implant.

The soft but fitting nature of the material allows a closed fitting onto the surface of the abutment and the implant as well as onto the surrounding soft and bone tissue.

This proximity in combination with the bone and soft tissue conductive properties will allow a fast and effective incorporation of the abutment.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A dental implant assembly comprising:
an implant;
an abutment; and
a shaped block formed of a dried cross-linked collagen matrix;
wherein said shaped block fits onto and partially covers an outer surface of the abutment and at least partially covers an outer surface of the implant when the abutment is placed into the implant; and
wherein the shaped block is configured to form a base for soft tissue ingrowth therein.

2. The dental implant assembly of claim 1, wherein the dried cross-linked collagen matrix further includes a non-polymeric sugar, and wherein the shaped block is retained by at least one of the abutment and the implant.

3. The dental implant assembly of claim 1, wherein the dried cross-linked collagen matrix is a non-polymeric sugar and further includes at least one of hydroxyapatite, titanium, and a pharmaceutically active agent.

4. The dental implant assembly of claim 3, wherein the pharmaceutically active agent comprises antibacterial agents, antifungal agents, anti-inflammatory agents, antibiotic agents, vitamins or any combination thereof.

5. The dental implant assembly of claim 1, wherein the shaped block is configured to form the base for both of the soft tissue ingrowth and a hard tissue ingrowth therein.

6. The dental implant assembly of claim 1, wherein the shaped block is shaped as an O-ring, sleeve or tube.

7. (canceled)

8. The dental implant assembly of claim 1, wherein the shaped block is configured for cell ingrowth and has a porosity of between 10-90%, and wherein the shaped block is configured for partial or full contact with bone or soft tissue when implanted.

9. A method of preparing a shaped block for a dental implant assembly, the method comprising:
(i) providing an acidic solution of collagen and a non-polymeric sugar crosslinker, followed by neutralization of the acidic solution;
(ii) concentrating the acidic solution of step (i) and pouring it into a mold with a predesigned block shape;
(iii) lyophilizing the acidic solution after concentrating, thereby obtaining a dried collagen composition;
(iv) incubating the dried collagen composition with the non-polymeric sugar crosslinker, and a first solvent;
(v) washing the dried collagen composition after incubating with a second solvent;
(vi) lyophilizing the dried collagen composition after washing;
(vii) carving out the shaped block from the dried collagen composition; and
(viii) coupling an implant of the dental implant assembly with an abutment of the dental implant assembly, wherein the shaped block fits onto and partially covers an outer surface of the abutment and at least partially covers an outer surface of the implant when the abutment is placed into the implant, and wherein the shaped block is configured to form a base for soft tissue ingrowth therein.

10. The method of claim 9, wherein the shaped block includes a non-polymeric sugar that is selected from a group consisting of glycerose, threose, erythrose, lyxose, xylose, arabinose, ribose, allose, altrose, glucose, mannose, gulose, idose, galactose, and talose.

11. The method of claim 9, wherein the mold is cooled to a freezing temperature of between −10° C. to −190° C. prior to lyophilizing of step (iii).

12. The method of claim 9, wherein the first solvent and second solvent are independently selected from water, ethanol, saline, methanol, and phosphate buffer saline.

13. The method of claim 9, wherein the mold is shaped using 3D printing or cast molding, and wherein carving out is performed using at least one of a CNC machine, a laser cutting machine, a waterjet cutter, a driller, and an abrasive device.

14. The method of claim 9, wherein the neutralization of the acidic solution uses a hydroxyapatite.

15. A dental implant assembly comprising:
an implant;
an abutment coupled to the implant; and
a shaped block formed of a dried cross-linked collagen matrix;
wherein the shaped block is configured as a sleeve, o-ring, or tube and fits onto and covers a portion of an outer surface of the abutment and extends to at least partially cover an outer surface of the implant; and
wherein the shaped block is configured for soft tissue ingrowth therein.

16. The dental implant assembly of claim 15, wherein the dried cross-linked collagen matrix is a non-polymeric sugar and further includes at least one of hydroxyapatite, titanium, and a pharmaceutically active agent.

17. The dental implant assembly of claim 15, wherein the shaped block is retained by at least one of the abutment and the implant, and wherein the shaped block is configured to form a base for one or both of soft tissue and hard tissue ingrowth therein.

18. The dental implant assembly of claim 15, wherein the shaped block is configured to form a base for one or both of soft tissue and hard tissue ingrowth therein and has a porosity of between 10-90%, and wherein the shaped block is configured for partial or full contact with bone or soft tissue when implanted.

19. The dental implant assembly of claim 15, wherein the dried cross-linked collagen matrix further includes a non-polymeric sugar.

20. The dental implant assembly of claim 15, wherein the shaped block is configured for both of the soft tissue and a hard tissue ingrowth therein.
